(19) United States
(12) Patent Application Publication
(10) Pub. No.: US 2026/0268278 A1
(43) Pub. Date: Sep. 10, 2026

(54) COLLABORATION PLATFORM FOR ANALYZING LARGE PRIVACY-IMPACTED DATASETS

(71) Applicants: Roche Sequencing Solutions, Inc., Pleasanton, CA (US); Roche Molecular Systems, Inc., Pleasanton, CA (US)

(72) Inventors: Carolina Dallett, Oakland, CA (US); Arick Huensche, Davis, CA (US); Victor Sundaram Sankarlingam, Barto, PA (US); Sowmithri Utiramerur, Pleasanton, CA (US)

(21) Appl. No.: 19/638,897

(22) Filed: Apr. 3, 2026

Related U.S. Application Data
(63) Continuation of application No. PCT/US2024/050537, filed on Oct. 9, 2024.
(60) Provisional application No. 63/589,914, filed on Oct. 12, 2023.

(51) Int. Cl. G06Q 10/101; G06F 16/21; G06F 21/62

(52) U.S. Cl. CPC G06Q 10/101; G06F 16/211; G06F 21/6209; G06F 2221/2141

(57) ABSTRACT

A collaboration platform is provided to analyze one or more biomedical data sets. The collaboration platform may include a project management engine, a data ingestion engine, and a data analysis engine. The project management engine may generate a plurality of project environments for analyzing biomedical data sets of interest, and designate a set of authorized users with access to given project environments. The data ingestion engine may process data sets and store them in a central data storage system for access within the project environments. The data analysis engine may perform analyses and special computing on the ingested data and generate updated output data that may be re-ingested for further analyses in the project environments.

### CROSS REFERENCES TO RELATED APPLICATIONS

The present application claims the benefit of PCT Application No. PCT/US2024/050537, which claims priority to U.S. Provisional Application No. 63/589,914, filed Oct. 12, 2023, the entire contents of each of which is herein incorporated by reference.

### TECHNICAL FIELD

The subject matter described herein relates generally to data management, database functionality, and more specifically, to biomedical database management, access, and processing.

### BACKGROUND

Biomedical data from individuals can be transformed into analytical data that when properly aggregated fuels machine learning models, trends biological historical perspective, and helps in identification of biomarkers that contribute to predictive analytics for disease diagnosis and treatment. In view of the interest in harnessing this potential power of biomedical data, biomedical data modalities are constantly changing and becoming increasingly more complex. At the same time, biomedical data are subject to regulations (such as privacy regulation) that can greatly vary depending on the geographical jurisdiction and use case. As a result of this variance in regulation, traditional approaches in managing biomedical data result in increasingly siloed data platforms. Such siloed data platforms reduce access to potentially useful data, which in turn reduces the effectiveness of the analysis, and slows down the process of gaining important insights on disease diagnosis and treatment. Thus, there is a need for a new data platform to allow for increased access of biomedical data while maintaining the requisite regulatory safeguards.

### SUMMARY

Methods, and systems, including computer program products, are provided for collaborative data management and analysis.

In an aspect, a system for collaborative data analysis is provided. The system includes a project management engine, a data ingestion engine, and a user interface. The project management engine is configured to generate a plurality of project environments for analyzing a plurality of biomedical data sets of interest, designate a set of authorized users with access to a given project environment of the plurality of project environments, and provide access, in each of the plurality of project environments, to at least one data analysis engine for analyzing at least one of the plurality of biomedical data sets. The data ingestion engine is configured to receive a data manifest associated with at least one of the plurality of biomedical data sets of interest, receive the at least one biomedical data set of interest, ingest the at least one biomedical data set of interest based on the associated data manifest, and store the ingested data of the data set of interest in a data storage system for access within at least one of the plurality of project environments. The data manifest includes information associated with the at least one biomedical data set of interest. The user interface is accessible by any of the authorized users, and provides access to at least one of the project environments in order to cause analysis to be performed on at least one of the plurality of data sets of interest.

In some aspects, the data manifest may include metadata associated with the data set of interest, and ingesting the at least one biomedical data set of interest comprises validating that metadata associated with the data set of interest corresponds to metadata identified in the data manifest. In some aspects, upon validating the metadata associated with the data set of interest corresponds to metadata identified in the data manifest, the ingestion engine is configured to enrich the at least one biomedical data set of interest by assigning a unique identifier to the biomedical data set of interest, the unique identifier being usable to track the use of the at least one biomedical data set of interest within the project management engine.

In an aspect, the system may also include a data analysis engine configured to perform analysis on at least one of the plurality of biomedical data sets, generate output indicative of the the analysis, and store an updated data set based on the generated output. In such aspect, the data ingestion engine may be configured to ingest the updated data set and store the ingested updated data set in the data storage system for access within at least one of the plurality of project environments.

In an aspect, each of the plurality of project environments may include a controlled sub-environment with a high performance computing system and an uncontrolled sub-environment with a data analytics computing system. In another aspect, each of the plurality of project environments includes a designated data analysis engine and dedicated storage for storing copies of the ingested data.

In an aspect, each of the plurality of biomedical data sets is assigned to at least one data schema based on the data manifest, and the system further includes a data management module configured to manage the data schema existing in the system. For example, the data management module may be configured to create additional data schema, modify existing data schema, and track modifications of existing data schema as different versions of the schema.

In an aspect the system further includes a data catalog listing ingested data sets of interest and information associated with the ingested data sets of interest.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to biomedical data management and analysis, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a diagram illustrating an example of the current state of organizations handling data analysis with multiple partners;

FIG. 2 depicts a diagram illustrating a collaboration hub, consistent with some implementations of the current subject matter;

FIG. 3 depicts a diagram illustrating an example of a data collaboration platform, consistent with some implementations of the current subject matter;

FIG. 4 depicts system diagram illustrating an example of a data collaboration platform integrated with a client device, consistent with some implementations of the current subject matter;

FIG. 5 depicts a flowchart illustrating an example of a project creation process consistent with some implementations of the current subject matter;

FIG. 6 depicts a flowchart illustrating an example of a data ingestion process consistent with some implementations of the current subject matter;

FIGS. 7A-7B depict illustrative sequential screenshots of a user interface triggering creation of a data manifest consistent with some implementations of the current subject matter;

FIG. 8 depicts a flowchart illustrating an example of a data analysis process consistent with some implementations of the current subject matter;

FIG. 9 depicts components of an illustrative project environment created in a collaboration hub, consistent with some implementations of the current subject matter;

FIG. 10 depicts components of a data management system, consistent with some implementations of the current subject matter;

FIG. 11 illustrates an example computer system that may be utilized to implement techniques disclosed herein;

When practical, like labels are used to refer to same or similar items in the drawings.

### DETAILED DESCRIPTION

Aspects of the current subject matter are directed to a collaboration hub for analyzing biomedical data.

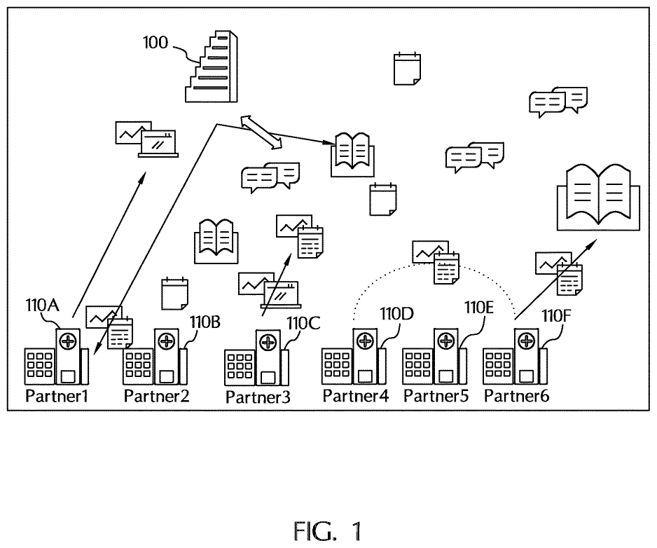

As described above, conventional systems for handling data analysis are often siloed, and do not allow for collaboration amongst numerous organizations. For example, it may be the case that an organization seeks to analyze biomedical data from a number of different research studies to gain insights and/or generate diagnostic tests and/or therapeutic treatment. In order to effectively carry out its research and gain actionable insights, the organization may need to work with multiple different entities to maximize the potential insights gained from the biomedical data. However, current systems do not allow this to be done collaboratively or efficiently. FIG. 1 is an illustrative depiction of how such an organization may currently operate. As can be seen in FIG. 1, organization 100 may have a number of partner organizations 110A-F that it collaborates with in the analysis of data. However, rather than a central system on which to collaborate, the interactions may all be siloed, where each organization 110 has different methods of sharing information, different formats for the information, different privacy safeguards, different computing/analysis tools, and so on. Thus, even when insights are gained with one partner that could be useful to build upon with other partners, there is often an inability to efficiently provide access to these insights for use in further analysis. This siloed approach results in inefficient analysis of data which slows the process of gaining important insights on such data.

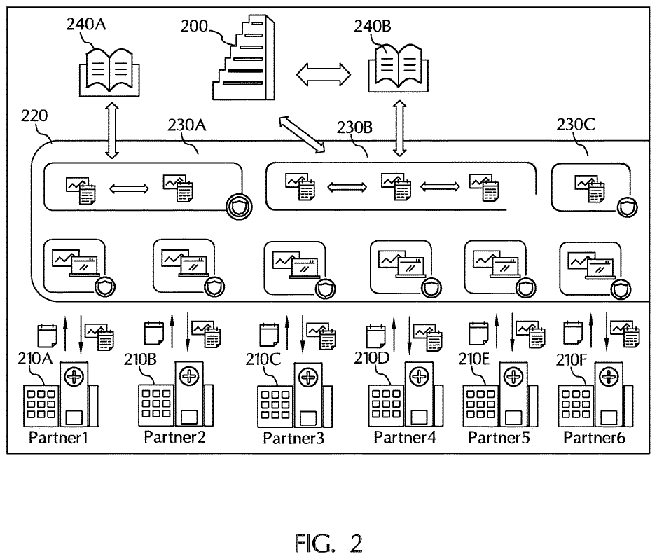

A collaboration hub, in accordance with implementations of the current subject matter, is thus provided to foster improved data and analysis sharing. An illustrative diagram of a collaboration hub is depicted in FIG. 2, which depicts how an organization 200 may utilize a centralized collaboration hub 220 to efficiently collaborate in sharing and analyzing data with any number of partner organizations 210A-F. As can be seen in FIG. 2, the collaboration hub 220 may host a number of data projects 230A, 230B, 230C which can be made accessible to any of partners 210A-F. Collaboration hub 220 may have the ability to obtain data from a number of external data sources 240A and 240B to be stored within the hub for access within the data projects 230 of the collaboration hub 220. And, as also can be seen in FIG. 2, each partner 210 may apply its data analysis tools and workflows on the data projects to perform analysis and append any insights it gains within the data projects. As will be explained below in more detail, collaboration hub 220 may be configured with a number of modules or engines that enable the efficient sharing of data and analysis thereof while maintaining regulatory constraints with respect to the data, such as those related to privacy, cybersecurity, and any other required safeguards.

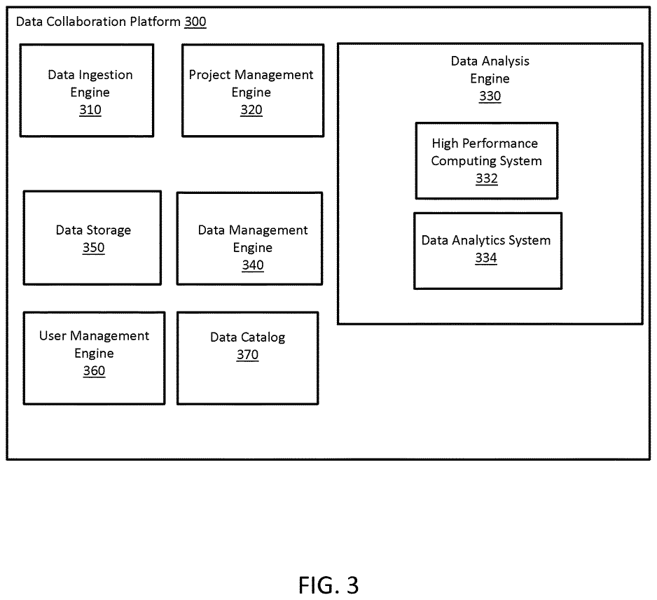

FIG. 3 depicts a diagram illustrating an example of a data collaboration platform 300, in accordance with implementations of the current subject matter. Data collaboration platform 300 may include various engines that, cooperatively, allow inter-organization collaboration in analysis of data, generation of insights, and development of analysis workflows to be applied on data, while maintaining important privacy, cybersecurity, and confidentiality safeguards on the data and insights.

The data collaboration platform 300 may include a data ingestion engine 310, a project management engine 320, a data analysis engine 330, a data management engine 340, a data storage system 350, a user management engine 360, and a data catalog 370. Data analysis engine may include or provide access to a high performance computing system 332 and/or a data analytics system 334. In some implementations, one or more aspects, features, and/or operations of the engines 310, 320, 330, 340, 350, 360 and/or 370 may be combined in various combinations.

Data ingestion engine 310 may manage the ingestion of data sets into the data collaboration platform 300 for access in various data analysis projects. Data ingestion engine 310 may receive as an initial input, for each data set, a previously ingested data model version, a data manifest detailing required information associated with the data set including clinical metadata, data generation pipeline information and any sample preparation information collected to manage data quality, and one or more files containing the data set(s) to be ingested. Data ingestion engine 310 may validate the data against the data manifest, and, if validated, cause the data to be stored in data storage system 350. Depending on the data type being ingested, the system automatically classifies data as “controlled” (example: fastq files) or “Open” (csv, txt). In some cases, the data manifest may also refer to a data-use model to designate organization-led nomenclature. Data ingestion engine 310 may enrich the data with additional information upon storage. For example, data ingestion engine 310 may generate and assign unique identifiers to each data object allowing file-name flexibility while enabling tracking and referencing uniquely the data, ensuring only authorized users are able to use a given data set. Once stored in the data storage system 350, data sets may be referenced in data catalog 370 and made available for use in data projects, where authorized. In case the data is not validated against the data manifest for any reason, data ingestion engine 310 may generate an error notification to the relevant user(s) and set the data manifest and data set aside for further input from a user. As will be described in more detail below, in addition to ingesting new data sets, data ingestion engine 310 may also ingest metadata associated with data sets to be ingested in the future, may re-ingest data sets with appended information generated by analysis thereof, and/or may ingest updated metadata to be associated with previously ingested data sets.

Project management engine 320 may be configured to manage all project activities such as creation and deletion of projects and services, compute settings and resources. Project management engine 320 may receive a request for creation of a project, along with any required information for creation. For example, project management engine 320 may, in response to a request for creation of a project, prompt a user to provide information such as the project name, an identifying code associated with the project (e.g., a work-breakdown structure (WBS) or other charge code), a geographical region where the project is hosted, and one or more requested services to be utilized by the project. Examples of requested services may include, but are not limited to, data analytics tools, computing platforms, code repositories, or other user-defined compute infrastructure such as high-performance computing (HPC), on-demand instances, databases, Jupyther notebooks, R-studio, Machine-learning tools (e.g. Sagemaker), storage capacity, and cost monitoring. In response to the request, project management engine 320 may generate the requested project as an isolated environment within collaboration platform 300, only available to authorized users of a given project. For example, project management engine 320 may generate a virtual private cloud (VPC) within the collaboration platform 300 for each requested project. Project management engine 320 may populate the project environment with designated data storage, and, depending on the requested services, one or more data analysis systems for analyzing the data. As will be described in more detail below, project management engine 320 may populate multiple data storage locations with different purposes. For example, project management engine 320 may designate one or more data storage locations for storing temporary copies of ingested data, one or more data storage locations for storing data analysis systems, and one or more data storage locations for storing output files generated in the process of analyzing ingested data.

In addition to creation of projects, project management engine 320 may be configured to delete projects. In some cases, project management engine 320 may receive a request from a user to delete a project. If the user is authorized to make such a request, the project management engine 320 may carry the request out. In some cases, the project management engine 320 may, in response to a project deletion request from an authorized user, prompt the user to also carry out deletion of all users associated with the project to be deleted. Upon confirmation, project management engine 320 may carry out deletion of the project and associated users from collaboration hub 300. In some instances, project configuration may be maintained in a database and may be used to re-establish a closed project as required.

Project management engine 320 may also manage authorized users for a given project, including adding authorized users, removing unauthorized users, and/or modifying a role associated with a given user. For example, an authorized user (such as one with “admin” status) may access the project management engine 320 to cause the engine to assign and remove users from a given project, and may modify the role of each authorized user (and thus control what access the user has within the project). User roles will be described in further detail below, in connection with user management engine 360. Data user management can be integrated with existing user active directories (e.g. LDAP) and own directory (for onboarding of externals).

The data analysis systems described above may be cloud-based computing systems enabled and managed by data analysis engine 330. For example, data analysis engine 330 may provision one or more dedicated high performance computing (HPC) systems 332 and/or one or more on-demand data analytics systems 334 for dedicated use within each project environment. Examples of high performance computing systems enabled by data analysis engine 330 include, but are not limited to, analysis orchestration engines such as Sagemaker, Nextflow, Cromwell, airflow, lambda functions running open-source and internal code developed over the years in on-premise solutions.

Examples of data analytics systems 334 include, but are not limited to, various combinations of CPUs and GPUs. Exemplary components are listed in Table 1 below. Although illustrative high performance computing systems and data analytics systems are described herein, it will be understood that these examples are not limiting, and any suitable computing systems and data analytics systems may be provided or accessed by data analysis engine 330.

TABLE 1
Exemplary data analytics systems

| | | | |
|---|---|---|---|
| CPU | Memory | Network | GPU |

General Purpose

| | | |
|---|---|---|
| 8 | 32 | 10 Gbps |
| 16 | 64 | 10 Gbps |

Compute Intensive

| | | |
|---|---|---|
| 8 | 16 | 10 Gbps |
| 16 | 32 | 10 Gbps |
| 64 | 128 | 10 Gbps |

Memory Intensive

| | | |
|---|---|---|
| 8 | 64 | 10 Gbps |
| 16 | 128 | 10 Gbps |
| 48 | 384 | 10 Gbps |
| 64 | 512 | 10 Gbps |

GPU Workloads

| | | | |
|---|---|---|---|
| 96 | 1024 | 20 Gbps | 8 GPUs/640 GB Memory |

Batch Computing

| | | |
|---|---|---|
| 2000 | 6000 | 20 × 10 Gbps |
| 4000 | 12000 | 40 × 10 Gbps |

Compute Templates
Hardened and Secured Compute Images
Deep Learning Compute Images
Containers
Deep Learning Containers

Project management engine 320, in coordination with data analysis engine 330, may configure the settings of the data analysis systems provisioned for a given project environment. For example, project management engine 320, may receive, at the time of project creation or otherwise, a request from an authorized user for installation of particular computing systems, data analytic systems, or other tools within a given project environment. If authorized and available, project management engine 320 may provision the requested system or tool on designated storage within the project environment. Project management engine 320 may also manage the designated storage within a project environment. For example, project management engine 320 may provision an initial amount of storage in separate file systems for storage of working files in the project environment, and monitor the usage of designated storage within a project environment. In some cases, upon reaching a threshold percentage of the designated storage, project management engine 320 may automatically provision additional storage to the project environment. For example, project management engine 320 may automatically provision additional storage when approximately 80% of the storage has been used. Such feature allows cost control and incentivizes users to ingest final analyzed versions of data and actively clear up unused/intermediate files.

Data Management Engine 340 may manage domain level changes related to the data sets available in collaboration hub 300. Domain level changes may be regarded as changes in the data schema, which refer to the different prescribed structures of the metadata. As described above with respect to data ingestion, each ingestion is accompanied by a data manifest, which identifies an applicable domain/scheme(s), and describes the metadata structure associated with said domain/scheme. Data management engine 340 may manage the different schema by allowing authorized users to view existing schema details, update existing schema to create new versions thereof, and create new schema entirely. For example, data management engine 340 may be configured to display, in response to a request from an authorized user, each of the active schema, as well as information associated with the active schema. Such information may include the domain/schema name, associated manifest ID, latest version number, the date created, and date modified. Data management engine 340 may allow for updating of existing schema by providing a template to a user, allowing the user to modify the template and upload the modified template, and updating the existing schema based on the modified template. Upon updating the existing schema, a new version of the schema may be generated, and may be made available as an optional domain/schema to be designated when ingesting data going forward. Data management engine 340 may also allow for creation of new schema by similarly allowing users to modify a template schema and upload the template in the system for creation by data management engine 340.

User Management Engine 360 may manage the authorized users of data collaboration platform 300, including on-boarding/creating new users, editing user details, and deleting users from the platform. In some implementations, only administrators of the collaboration platform 300 may employ user management engine 360 for the above operations. For example, an authorized user, such as a designated system administrator, may be able to view all active users of collaboration platform 300 and information associated with the users, such as usernames, contact information, organization information, and associated projects that the user has access to. Such authorized users may also be able to edit any of these aspects associated with a given user. An authorized user may also be able to create new users by inputting the relevant information. Finally, an authorized user may be able to remove or delete existing users.

Data Catalog 370 may be an index providing information and access related to various data objects that have been ingested into data collaboration platform 300. In some instances, data catalog 370 may provide limited information relating the type of data objects available, depending on who is accessing the catalog. As will be described below in further detail, data catalog 370 may provide an interface for authorized users to access one or more data sets for use in any authorized user's respective projects.

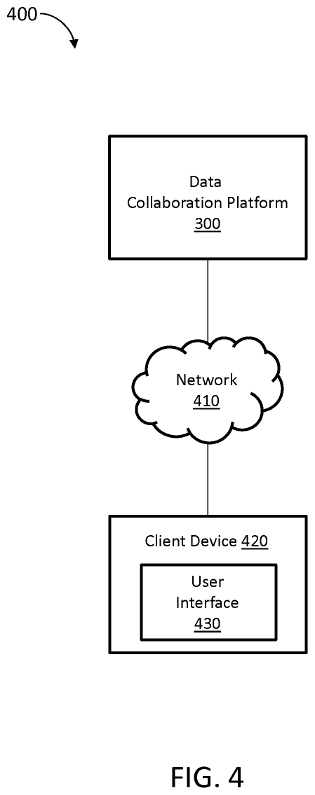

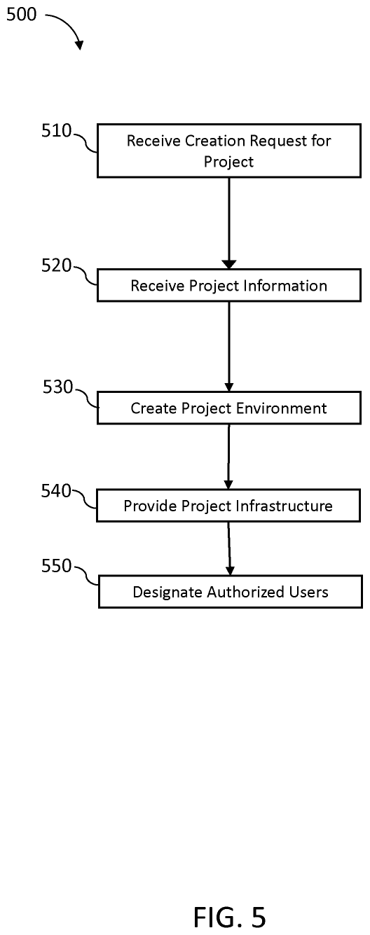

FIG. 4 depicts a system diagram illustrating an example of a collaboration platform integrated with a client device. With reference to FIG. 5, a data collaboration system 400 includes the data collaboration platform 300 and a client device 420. The data collaboration platform 300 and the client device 420 may be communicatively coupled via a network 410. The network 410 may be a wired network and/or a wireless network including, for example, a local area network (LAN), a virtual local area network (VLAN), a wide area network (WAN), a public land mobile network (PLMN), the Internet, and/or the like. The client device 420 may be a processor-based device including, for example, a workstation, a desktop computer, a laptop computer, a smartphone, a tablet computer, a wearable apparatus, and/or the like. The client device 420 may have a user interface 430 on which data generated by and/or communicated to the client device 420 may be displayed.

In some implementations, the data collaboration platform 300 may be hosted on a cloud-based infrastructure such that the functionalities of the data collaboration platform 300 are accessible remotely, for example, as part of a web-based application, a native mobile application, a software-as-a-service (SaaS), and/or the like. Moreover, each of the data ingestion engine 310, project management engine 320, data analysis engine 330, data management engine 340, data storage 350, user management engine 360, and data catalog 370 may be implemented as interchangeable plugin modules. As such, one or more of the data ingestion engine 310, project management engine 320, data analysis engine 330, data management engine 340, data storage 350, user management engine 360, and data catalog 370 may be replaced with alternatives in order to provide different and/or additional functionalities.

The client device 420, with the user interface 430, allows for user interaction with respect to the data collaboration platform 300. For example, a clinician/researcher or other user may initiate the data collaboration platform 300 to create a project, ingest data, and perform analysis thereon, which may be shared with other users. Information and/or data associated with the project's own data sets, authorized external data mounts and/or projects may be generated and displayed on the user interface 530.

The data collaboration platform 300 and/or the client device 420 may be communicatively coupled to one or more other platforms, servers, and/or devices via, for example, the network 410 or another network. The data collaboration platform 300 and/or the client device 420 may then provide the results of the data analysis as performed by the data collaboration platform 300 to the one or more other platforms, servers, and/or devices.

It can be seen that the various engines of data collaboration platform 300 may cooperatively allow inter-organization collaboration in analysis of data, generation of insights, and development of analysis workflows to be applied on data, while maintaining important privacy, cybersecurity, and confidentiality safeguards on the data and insights. However, it may be desirable that not all users have access to each of the engines described above. Accordingly, it may be desirable to assign roles to each user which designate what level of access and control a given user has within a data collaboration platform. An exemplary designation of such roles and level of access and control is provided in table 2 below:

TABLE 2
Exemplary Access and Actions available by User Role

| | | | | | Data | Data | |
|---|---|---|---|---|---|---|---|
| | | Platform | Data | Privacy | User | User | Unassigned |
| Module | Action | Admin | Manager | Officer | I | II | User |
| Dashboard | User Management | x | | | | | |
| | Project Management | x | x | x | x | x | x |
| | Data Catalog | x | x | x | x | x | x |
| | Data Management | x | x | | x | | |
| | Important Links | x | x | x | x | x | x |
| User | View platform users list | x | | | | | |
| Management | Create User | x | | | | | |
| | Edit User | x | | | | | |
| | Delete User | x | | | | | |
| | Modify user's platform | x | | | | | |
| | specific role | | | | | | |
| Project | View list of all projects on | x | | | | | |
| Management | platform | | | | | | |
| | View list of specific | | x | x | x | x | |
| | projects assigned to him | | | | | | |
| | Create new project | x | | | | | |
| | Delete Project | x | | | | | |
| Project | “Project Access” option | | x | x | x | x | |
| Management - | View users in the project | x | x | x | x | x | |
| Project | Assign/Remove user | x | x | | | | |
| Access | to/from project | | | | | | |
| | Edit user role in project | x | x | | | | |
| Project | “Technical Settings” option | x | x | | x | x | |
| Management - | | | | | | | |
| Technical | | | | | | | |
| Settings | | | | | | | |
| | Project Settings - View | x | x | | x | x | |
| Technical | View all info on page | x | x | | x | x | |
| Settings - | Request computing | x | x | | x | x | |
| HPC system | instance | | | | | | |
| settings | View the instances | x | x | | x | x | |
| | requested by all users in | | | | | | |
| | project | | | | | | |
| | View the instances | x | x | | x | x | |
| | requested by self in project | | | | | | |
| | Terminate instance | x | x | | x | x | |
| | requested by self | | | | | | |
| | Terminate instance | x | x | | | | |
| | requested by others in the | | | | | | |
| | project | | | | | | |
| | Start a session on project's | x | x | | x | x | |
| | HPC instances | | | | | | |
| | Pull code and images from | x | x | | x | x | |
| | CodeCommit and ECR | | | | | | |
| | (HPC system) | | | | | | |
| | Push code and images to | x | x | | x | x | |
| | CodeCommit and ECR | | | | | | |
| | (HPC system) | | | | | | |
| Technical | View all info on page | x | x | | x | x | |
| Settings - | Request computing | x | x | | x | x | |
| Analytics | instance | | | | | | |
| system | Access RStudio | x | x | | x | x | |
| settings | View the instances | x | x | | x | x | |
| | requested by all users in | | | | | | |
| | project | | | | | | |
| | View the instances | x | x | | x | x | |
| | requested by self in project | | | | | | |
| | Terminate instance | x | x | | x | x | |
| | requested by self | | | | | | |
| | Terminate instance | x | x | | x | x | |
| | requested by others in the | | | | | | |
| | project | | | | | | |
| | Start a session on project's | x | x | | x | x | |
| | Analytics system instances | | | | | | |
| | Pull code and images from | x | x | | x | x | |
| | CodeCommit and ECR | | | | | | |
| | (Analytics system) | | | | | | |
| | Push code and images to | x | x | | x | x | |
| | CodeCommit and ECR | | | | | | |
| | (Analytics system) | | | | | | |
| Technical | View Project specific | x | x | | x | x | |
| Settings - | repositories | | | | | | |
| Code | Request for Global Access | x | x | | x | x | |
| Repositories | View Awaiting Approval | x | | | | | |
| | page | | | | | | |
| | View Global access | x | | | | | |
| | requests | | | | | | |
| | Approve or Reject Global | x | | | | | |
| | access requests | | | | | | |
| Project | “Data Ingestion | x | x | | x | | |
| Management - | Management” option | | | | | | |
| Data Ingestion | Re-ingest files | x | x | | x | | |
| | View the manifest list | x | x | | x | | |
| | Download Manifest | x | x | | x | | |
| | Template from UI | | | | | | |
| | Create Manifest/Metadata | x | x | | x | | |
| | from UI | | | | | | |
| | Edit Manifest/Metadata | x | x | | x | | |
| | from UI | | | | | | |
| | Delete Manifest/Metadata | x | x | | x | | |
| | from UI | | | | | | |
| | Save Manifest/Metadata in | x | x | | x | | |
| | platform local storage | | | | | | |
| | Cancel Manifest/Metadata | x | x | | x | | |
| | creation from UI | | | | | | |
| | Download | x | x | | x | | |
| | Manifest/Metadata to local | | | | | | |
| | system | | | | | | |
| | Submit Manifest/Metadata | x | x | | x | | |
| | for Ingestion | | | | | | |
| | Direct drop files/Manifest | x | x | | x | | |
| | into ingest S3 folders | | | | | | |
| | Upload Manifest through | x | x | | x | | |
| | CSV | | | | | | |
| Data | Data Management App | x | x | | x | | |
| Management | View Global and Domain | x | x | | x | | |
| | Schema tabs | | | | | | |
| | Download global and | x | x | | x | | |
| | domain schema based on | | | | | | |
| | versions | | | | | | |
| | Create new domain | x | x | | | | |
| | schema | | | | | | |
| | Update existing domain | x | x | | | | |
| | schema | | | | | | |
| | Update global schema | x | x | | | | |

As can be seen in the table, roles may include “Platform Admin,” (assigned to users with no access to data, but able to manage the platform) “Data Manager,” “Privacy Officer,” “Data User I,” “Data User II,” (the project user roles) and “Unassigned User”. Any unassigned user may be assigned different roles in different projects. Depending on the role, different modules (corresponding to the engines as described above with reference to FIG. 3) may be visible to that user in his or her dashboard when accessing the collaboration platform from a client device via the user interface, and within each module, different actions may be available to the user. While Table 2 provides exemplary designations for exemplary actions, it will be appreciated that any suitable relationship may be designated between a given user's role and the access and actions available. Moreover, additional roles may be created with different levels of access and control and additional actions may also be contemplated, in accordance to implementations discussed herein.

As described above, the core functionalities of the collaboration platform described herein include creation of discrete project environments with controllable access, ingestion of data according to standard and customizable schema for access within project environments, and performance of data analysis on the ingested data within project environments in a way that allows for controlled yet shareable output to allow for efficient collaboration and aggregating of insights. Consistent with implementations discussed herein, project creation, data ingestion, and data analysis will be discussed in further detail below.

### Project Creation

Before data can be ingested and analyzed, one or more project environments need to be created. Project creation will be described in greater detail with reference to FIG. 5, which depicts a flowchart illustrating an example of a project creation process 500 consistent with implementations of the current subject matter. Referring to FIG. 3, process 500 may be performed by the collaboration platform 300, including project management engine 320 along with any combination of the components of collaboration platform 300.

At 510, a request for a project creation is received. The request for project creation may be made by a user, such as the platform administrator (“platform admin”) through the user's project management dashboard. Once an initial request for creation of a project is received, for example, by the project management engine 320, the engine may prompt the user for the required information for project creation.

At 520, project information is received. As noted above, project information may be received by the user in response to a prompt generated by the project management engine 320. Required project information may include a project name, an identifying code (such as a charge code), a region associated with the project, storage and compute requirements, and specially requested services and software required for the project. As noted above, requested services required for the project may include specific requests for computing resources (such as HPC configurations), data analytic (DA) instances like CPUs and GPUs, links to specific external databases, or any other suitable services or systems that may aid in analysis of data sets within the project environment. For example, projects may be configured to have installation of software applications that help programmers develop software code efficiently such as an integrated development environments (IDEs) and other developer platforms (e.g. AI platforms such as Weights and Biases or Sagemaker), Notebooks (e.g. Jupyter or R-markdown) and Docker registries. A project may also be configured to have a duration date and level of data management enforcement. For example, some projects may be spun out for secondary analysis without any data ingestion needed to be enforced, while other projects may be configured to have more restrictive control of the data and analysis thereof.

At 530, a project environment is created in response to the creation request and project information received at 510 and 520 respectively. For example, the project management engine 320 may generate the requested project as an isolated environment within collaboration platform 300. Each generated project environment may only be made available to authorized users of a given project, which will be described further with respect to step 550. In some instances, project management engine 320 may create each project environment as a virtual private cloud (VPC) within the collaboration platform 300. This effectively creates a clean environment which multiple organization users may use in a collaboration setting.

At 540, the project infrastructure is provided in the project environment. The project management engine 320 may provide certain infrastructure by default and certain additional infrastructure based on the project information received at step 520. For example, each project environment may include storage for data to be analyzed, and certain computing resources for analyzing the data. In some instances, the computing resources provided by the project management engine 320 may include one or both of high performance computing system 332 and data analytics system 334. Depending on the computing resources requested, additional infrastructure such as dedicated storage for the computing resources may also be provided by project management engine 320. In some instances, one or more docker repositories and code repositories may be provided within a project environment. For example, there may be default code repositories provided for all projects, there may be region-specific code repositories, there may be a library of additional code repositories that can be made available upon request, and there may be additional user created code repositories that can be stored in a given project upon request. Code repositories may be used to track versions of code, track analysis and code action, share pipelines, and reference pipelines during analysis orchestration. Code can be shared with project users or platform users.

In some instances, project management engine 320 may provide multiple sub-environments within a project environment, where each sub-environment has varying restrictions or controls. For example, the system may provide two sub-environments for users, including a first highly controlled high performance computing environment without any internet access to prevent unintentional egress of data and a second general data analytics environment with more flexibility and connectivity. Provision of such dual environments may foster code development consistent with how users normally perform such tasks, allowing for external data and code mounts via internet access but controlling the data check-out to the controlled environment when code can run. For instance, the user may utilize the more controlled environment (with an HPC) to run heavy load bioinformatics analysis and use the less controlled data analytics (DA) environment to load a personally assigned compute instance with additional tools (such as Jupyter notebooks, R-studio, etc. as described herein) to perform statistical analyses, machine learning, and/or apply the user's existing data analytics pipeline on the data sets.

At 550, authorized users may be designated for the newly created project. For example, once a project is created, an authorized managing user (such as a platform admin or data manager) may identify users to be assigned to the created project and the project management engine 320 may add the assigned users as authorized users of the project. In addition to identifying users to be assigned to a given project, the managing user may assign specific roles to the users, such as one of the roles described with respect to Table 1 above. Once the request for authorized users and their roles is received by the project management engine 320, the users may be given the access associated with their roles with respect to the created project. Each of the users assigned to the project may then see the created project when accessing the user's project management dashboard, and may have access to the various functions associated with their respective roles.

### Data Ingestion

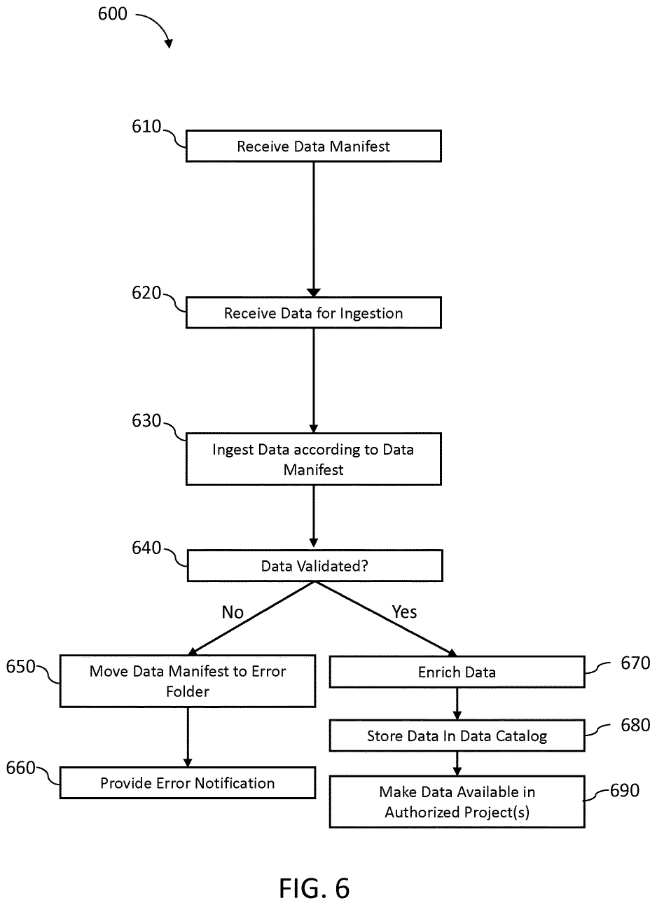

Once project environments have been created, data may be ingested into the collaboration platform so that it can be made available for use within the project environments. Data ingestion will be described in greater detail with reference to FIG. 6, which depicts a flowchart illustrating an example of a data ingestion process 600 consistent with implementations of the current subject matter. Referring to FIG. 3, process 600 may be performed by the collaboration platform 300, including data ingestion engine 310 along with any combination of the components of collaboration platform 300.

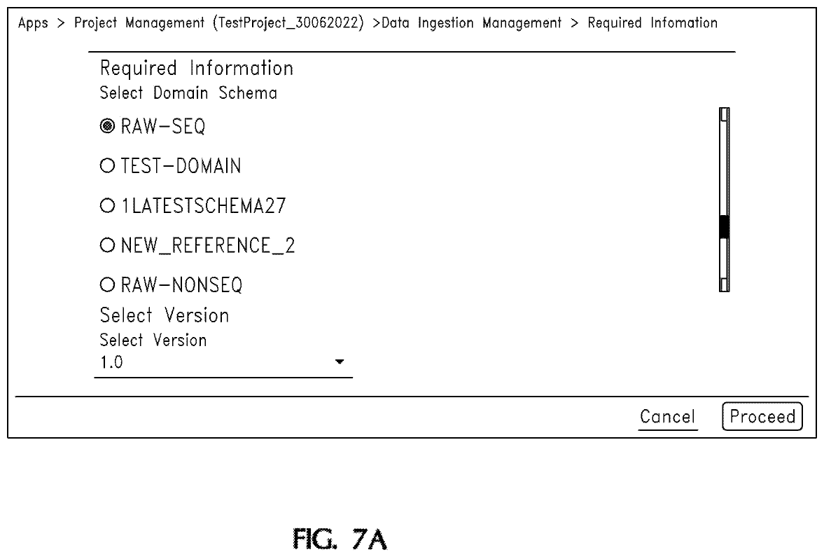

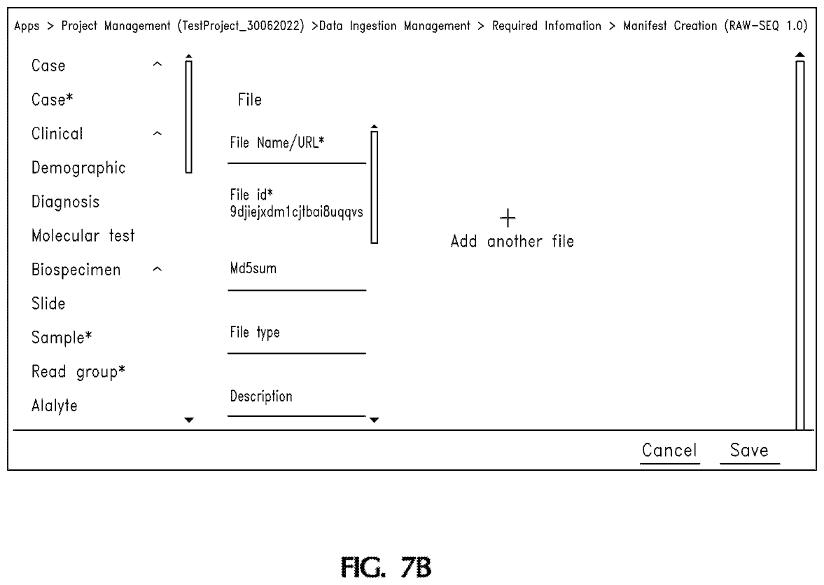

At 610, the data ingestion process begins with the receipt of the data manifest. In some instances, the data ingestion engine 310 may prompt the user to input information to generate the data manifest. For example, the data ingestion engine 310 may respond to a request from a user to create a data manifest by requesting information (via a user interface) from the user that triggers the creation of the data manifest. FIGS. 7A-7B depict illustrative screenshots of a user interface that may trigger creation of a data manifest consistent with implementations of the current subject matter. For instance, the data ingestion engine 310 may prompt a user to select a domain (or schema as described above) associated with the data file that is to be ingested, along with a version number of the domain as shown in FIG. 7A. In response to the selected domain/schema and version number, the data ingestion engine 310 may generate a manifest creation page populated with required fields associated with the selected version of the selected domain/schema as shown in FIG. 7B. Once the required fields of the manifest have been filled in with information by the user, the manifest may be saved and received by the data ingestion engine 310.

Although described above in terms of a user interface pre-populated by the data ingestion engine 310 and supplemented with user input, the data manifest may also be uploaded in a formatted file with the requisite information filled out. For example, data ingestion engine 310 may generate a manifest file to be downloaded with marked required information, and may provide a location for the completed manifest file to be uploaded to initiate ingestion.

At 620, the ingestion engine starts once the data manifest and its reference data files are received. If the manifest is provided through the user interface as described above, the data file may be uploaded using the same user interface, by linking to the location of the data file to be ingested, or otherwise uploading the data file to be ingested into the designated area for ingestion of data. In some instances, multiple data files may be ingested using the same manifest. In some instances, a pre-signed url may be referenced in the manifest and that valid link may also trigger the data ingestion protocol.

At 630, once it is confirmed that the data manifest and data to be ingested have been received, the ingestion of the data may proceed according to the data manifest. Data ingestion engine 310 may compare the data file to the data manifest to confirm that the data file conforms with the metadata instructions laid out in the data manifest. At 640, the data is validated against the data manifest. If the data is incomplete or otherwise does not match what is expected by the data manifest, or if another error is present in the data file and/or data manifest, the data ingestion engine 310 deems the data not validated, and does not proceed with ingestion. Instead, data ingestion engine 310 proceeds to step 650, where it moves the data manifest into an error folder for further processing, and proceeds to step 660 where an error notification is provided to the user. The error notification may provide details regarding the error, such as the location in the data file of the error, and details of the error so that the user can remedy the errors. The user may update either the data manifest or data file and attempt to ingest the data again.

If the data is successfully validated against the data manifest at step 640, ingestion proceeds with the data being enriched at step 670. The data may be enriched by assigning unique identifiers to the data for use in auditing, privacy, and cybersecurity. If not already referenced, clinically relevant fields such as patient_id, sample_id and aliquot_id are also assigned unique identification. This unique identification will follow the data throughout its use in data collaboration platform, such that users with access will be able to track the data and its use throughout its lifecycle in the platform. To that end, any action taken with the data, including the copying, analysis, re-ingestion, updating, or other actions, may be logged and associated with the unique identification. Once enriched, system administrators may elect to scan the data with anti-virus and PHI scanners, and then the data ingestion engine 310 proceeds to 680, where the enriched data is stored together with the data file in a data storage system. In some cases, upon storage of the data file, certain descriptive metadata associated with the data file can be viewed by some or all users in the data catalog 370. In some instances, data may be classified as controlled based on the type of data models used (for example those defining fastq files). Such classification may enforce and inform the users the data can only be available in high security project locations with limited to no internet access. In some instances, projects and any data ingested therein may take on additional classifications based on business rules (or any other suitable rules or restrictions to be applied) and such data may be marked as embargoed based on a project status and any other business rules defined at project creation. Data files may inherit the current access rules of the associated project (for example, embargoed project, open source data only, etc).

At step 690, the ingested data is made available in the relevant project environment. Data may be made available in the relevant project environment by storage of a temporary copy within the designated storage in the project environment. For example, once logged into the data catalog, a user can choose and “check out” a given data file, which will trigger a copy of the data to be stored in a designated storage location in the project environment. In some instances, a user may reference the unique id of a file and utilize an API for similar action from the analysis environment. Importantly, while the data is permanently stored (subject to any temporal or other restrictions on its use) in the data management storage and described in the data catalog, the data and full metadata is only visible to users associated with authorized projects. This granular control of the data access, while also making it readily available when appropriate, contributes greatly to the power of the data collaboration platform described herein.

### Data Analysis

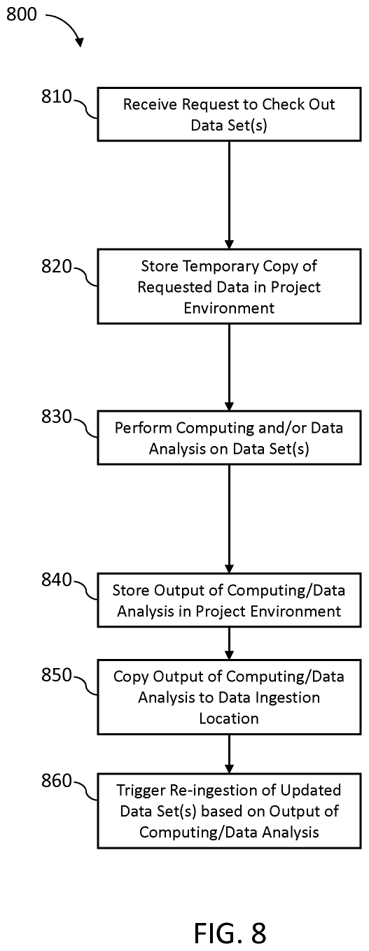

Upon creation of project environments and ingestion of one or more data sets for use within one or more project environments, the data may be analyzed to generate insights and output for further analysis. Data analysis will be described in greater detail with reference to FIG. 8, which depicts a flowchart illustrating an example of a data analysis process consistent with implementations of the current subject matter. Referring to FIG. 3, process 800 may be performed by the collaboration platform 300, including data analysis engine 330 along with any combination of the components of collaboration platform 300.

At 810 a request may be received to check out one or more data sets for use within an authorized project environment. For example, an authorized user for a given project may access the project management dashboard and request to check out one or more ingested data sets for use in a project environment. As another example, an authorized user may search the data catalog for one or more data sets and submit a request to check out said data sets into a given project environment for further analysis.

At 820, provided that the user and project are authorized to access the requested one or more data sets, a temporary copy of the requested data set may be stored in a designated storage of the project environment as read-only. As noted above, the act of checking out the data set may be logged by the system for audit and other tracking purposes, and may be stored to an audit trail that is accessible by, for example, a privacy officer or other suitable role.

At 830, an authorized user assigned to the project environment may perform analysis of the checked out data sets within the project environment. For example, the user may utilize any of the infrastructure provided in the project environment as described above, to perform computing and/or analysis of the data. As described above, the system may provide two sub-environments for users, a highly controlled environment without any internet access to prevent unintentional egress of data and a general off-the-shelf data analytics environment with more flexibility and connectivity. For instance, the user may utilize the more controlled environment (with an HPC) to run heavy load bioinformatics analysis and use the less controlled data analytics (DA) environment to load a personally assigned compute instance with additional tools (Jupyter notebooks, R-studio, etc) to perform statistical analyses, machine learning, and/or apply the user's existing data analytics pipeline on the data sets. The computing and or analytics may yield certain outputs associated with the data which may provide insights about the data that could be used to improve relevant diagnostics, improve development of treatments, or otherwise advance the analysis of the data. In such a case, and whenever an output is to be long-term stored, the output may be stored in the project environment at step 840. The output files may initially be stored in designated storage associated with the HPC or DA tools that generate the output. In some cases, the output may include an update to the initially ingested data, such as further appended information that was generated during the analysis performed at step 830. In some cases, the output may result in an updated data set that includes the original data set and any appended information gleaned during step 830.

Once the output is stored, it may be copied to a location designated for data ingestion at step 850. For example, a user seeking to re-ingest the updated data including any outputs generated during step 830 (and stored in step 840) may manually make a copy of the output in a location that is designated for ingestion of data in a given project environment.

Once the updated data (including any appended information gleaned during step 830) is available in a data ingestion location, the user may trigger re-ingestion of the updated data into one or more project environments for further use in data collaboration platform 300 at step 860. For example, the user may trigger re-ingestion of the updated data similar to the process 600 described above, by selecting the domain/schema, generating a data manifest perhaps automatically while manipulating the data, and submitting the updated data set for ingestion. In some cases, re-ingestion of certain files may not require a unique data manifest, if, for example, the files are accessory files that do not contain source data. As an example, this may include files generated for quality control.

As with any data ingested into collaboration platform, once re-ingested, the updated data may be used for further analysis within data collaboration platform 300 by any authorized users of the project. Thus, not only do users have the ability to share existing data across projects, but they have the ability to share insights gained on the data and continue to build upon each other's work. This centralized, collaborative approach makes the collaboration platform a powerful tool for efficiently and safely analyzing and sharing data with partners, in direct contrast with conventional methods as described above.

### EXAMPLES

In an illustrative example of process 800, users of a given project may be tasked with analyzing genomic sequencing data initially provided in the form of fastq files. The fastq files may be ingested for a given project, and users may utilize an internal tool or an external/open source tool (e.g. a pipeline written using any of the tools described herein) configured to glean certain features from the fastq files, such as single nucleotide polymorphisms (SNPs). Accordingly, users may utilize either the internal or external/open source tool to generate variant call format (VCF) files indicative of the SNPs in one of the sub-environments detailed above. Once the VCF files are generated, the user may cause them to be re-ingested along with any associated manifest (which may have been generated by the pipelines).

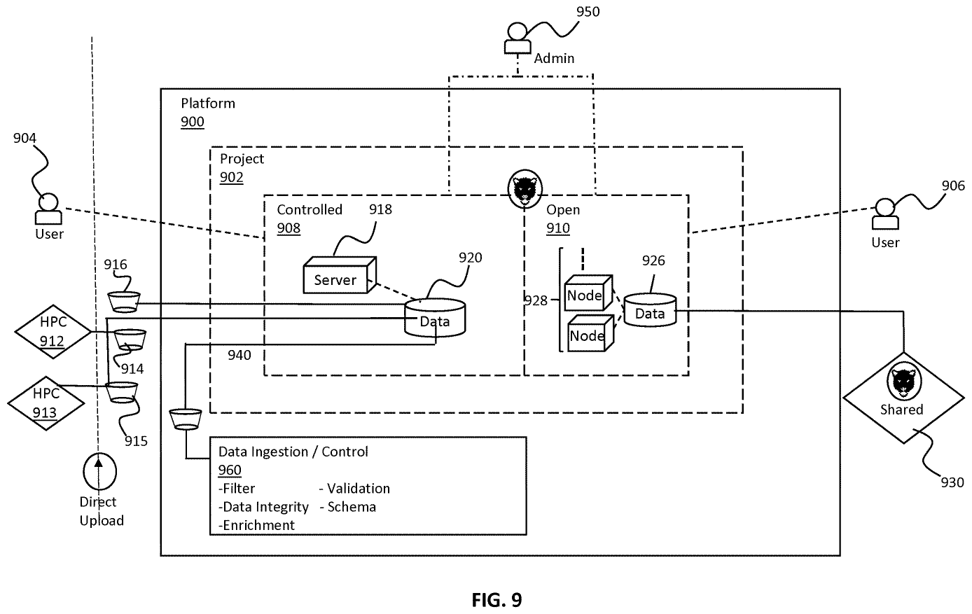

Another illustrative example will be described in more detail with reference to FIG. 9, which depicts components of an illustrative project environment 902 created in a collaboration platform 900, consistent with implementations of the current subject matter. In the illustrative example, a project user (e.g. 904 or 906) may want to perform analysis followed by machine learning with respect to data for a given project. As described with respect to the processes above, a project admin may create a project, designating a project environment 902 (as a VPC) with two sub-environments, a controlled environment 908 with an HPC system and an uncontrolled environment 910 with a DA system.

Controlled environment 908 may include computing resources which include a master server 918 and a number of computing nodes 920 coupled thereto. For example, the computing resources may include a cluster configured by the system admin 950 that includes a combination of cloud computing instances (e.g. Amazon Elastic Compute Cloud, or EC2) managed by a cloud native HPC management tool (e.g. AWS ParallelCluster and Slurm Scheduler). General purpose instances in the desired configurations can be evaluated for job size, cost of compute and turn-around-time optimization. Controlled environment 908 may also include additional user requested computing resources 922 based on specific user requests. For example, user requested computing resources 922 may include additional cloud computing instances and/or notebook tools (such as Jupyter). Controlled environment 924 may include dedicated storage 924 for storing data, which may include any suitable file systems (e.g., FSx) configured depending on the needs of the project. Additionally, any compute node assigned to the project may have automatically mounted one or more docker registry (e.g. AWS Container Registry) and source code repositories (e.g. CodeCommit) (generally depicted as 932 in FIG. 9).

Uncontrolled environment 910 may include user requested computing resources 926 which may include any cloud computing instances or other tools as described above, and dedicated storage 928, which may include suitable file systems configured based on the needs of the project as described above. As noted above, uncontrolled environment 910 may include access to whitelisted web addresses, which may include access to one or more code repository 930. Code repository 930 may be any suitable repository, including open source repositories, internal organization repositories, or other suitable repositories.

Once configured as described above, the project admin may onboard a number of users into the platform and assign at least one Data Manager to a project. The assigned Data manager may then assign at least one Data user I (e.g. 904 or 906). It may be desired to obtain data, such as sequencing data in the form of binary base call (BCL) sequence format from a partner laboratory or other external organization, but there may be a need to convert the files into a suitable format for performing the desired analysis. Accordingly, the BCL sequencing files may be moved to the cloud using an encrypted/secure connection (e.g. an sftp connection) from storage outside of platform 900 (such as an of storage 914, 915, 916) to a specific ingestion folder 934 created for the project. The admin may enable the mount of those folders into controlled system 940 as depicted by 916. In one example, a workflow (or pipeline) implemented using one or more tools stored on repository 932 may read the BCL files (received via storage 916 e.g.) and convert them into fastq files stored in storage 924. The workflow may further create manifests that allow for demultiplexing of the fastq files into various existing metadata fields (for example, patient/case_id, sample_id, aliquot_id) for use in ingesting the data further. In some cases, the fastq files are further processed by code stored in repository 932 to generate the desired output files, which may be VCF files which provide somatic mutation calls. The code used may include internal code stored on repository 932 and open source code that is first obtained in uncontrolled environment 910 at storage 928 via repository 930, and then pushed to the code commit repository 932 for access in controlled environment 908. The output VCF files and accompanying manifest may then be created by this code and copied to the ingestion folder 934 (via 940) and ingested by a data ingestion engine 960 as described in the processes above. Throughout this process, it can be understood that any number of intermediate files may be generated, some of which may be ingested using manifests, and some of which may not need manifests for ingestion, depending on the contents. For example, intermediate files may include “.bam” and index “.bam.bai” files which may need manifests for ingestion since they may include aspects from the original source data. On the other hand, some accessory files may simply be generated for quality control purposes and/or may not include source data, and thus may not require manifests (these may be, for example, txt, csv, xls, pdf, gif, ppt files).

Once the VCF files are ingested, Data User 906 may now enter the DA environment 910 and open a tool from resources 926 (such as RStudio or Jupyter Notebook) in the user interface which may open the tool with only the project mounted. Data User 906 may now checkout all VCF files to the DA environment together with the relevant metadata (e.g. case_id clinical metadata) and perform work reading these files for modeling and running predictions (e.g. using machine learning). When successful, the notebook can be versioned, tracked and saved, and any output, such as plots or other images can be exported and/or re-ingested. Data User 906 may then use IDEs to manipulate code and write notes, and create markdowns that can be used for documentation and publishing purposes. And, as studies continue, case longitudinal information may be updated further.

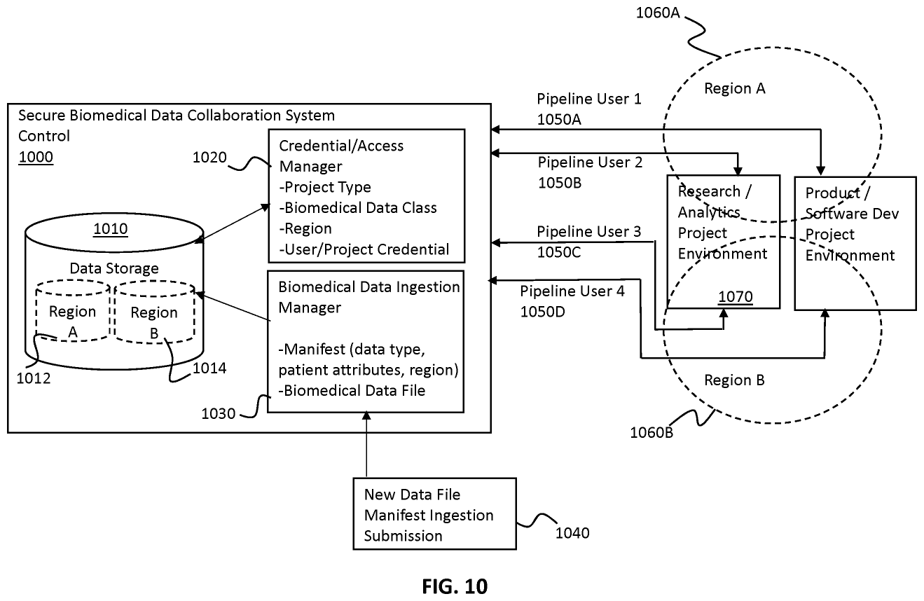

FIG. 10 depicts components of a data management system, consistent with some implementations of the current subject matter. A secure data management system 1000 includes a data storage system 1010, which may include interconnected data storage facilities and devices 1012 and 1014 in various geographical regions 1060A and 1060B. As further described herein, data files stored in particular regions (e.g., 1060A, 1060B) may be subject to particular security and/or privacy regulations (e.g., EU General Data Protection Regulation (GDPR)) and the system 1010 can be configured to manage content and access by users accordingly based on data properties (e.g., sensitive health information), by whom/where access is granted, project type (e.g., research/product development) and/or where the data is stored (e.g., EU, California). Data storage system 1010 may facilitate use of cloud computing services for storing and processing such data and may also include safeguards according to regional privacy/security laws.

An access manager component 1020 includes programming to control/manage access to data sets such as according to the types, regions, and uses applicable to the data sets. For example the access manager component 1020 may control access based on a project environment for collaborative data sharing with research institutions (e.g., a university) and limit access to particular portions of certain data sets (e.g., molecular sequences of certain proteins or pathogens), analysis or analytical models pertaining to the data sets, and/or an anonymized (or pseudo-anonymized) version of data in accordance with applicable regional laws.

In some embodiments, a project environment 1080 may be dedicated and configured for internal development of a product (e.g., a diagnostic molecular test, machine learning models) where access can be limited to users with particular credentials (e.g., classes of employees/contractors) and where the data may include aspects of sensitive diagnostic data and/or proprietary software development code. Data access pipelines 1050A and 1050D may be created through which users can access the data. Depending on the region 1060A or 1060B through which the data set is being accessed, different levels of access and control of the data through the pipelines may further be managed by system 1000.

In some embodiments, a project environment 1070 may be dedicated and configured for collaborative and relatively open research (e.g., spread of a disease or condition, data analytics, peer review) where access can be granted to members of a research institution and where the data set may include anonymized (or pseudo-anonymized) versions of the data or data analysis. In some embodiments, the project environment is dedicated to the creation and sharing of data models or analysis of the data. Data access pipelines 1050B and 1050C may be created through which users can access the data set. Depending on the region 1060A or 1060B through which the data set is being accessed, different levels of access and control of the data set through the pipelines may further be managed by system 1000.

A biomedical data integestion component 1030 includes programming to process requests for adding biomedical data sets (and/or metadata) for access/storage within system 1000. In some embodiments, a data manifest is obtained and used to process a data ingestion request. The data manifest can include data set attributes including, for example, a data type (e.g., molecular sequencing data), attributes of data subjects (e.g., patient characteristics), a region/location associated with the data (e.g., where the data originated or is to be stored within the system), and/or security classifications with which the data is to be managed. As further described herein, the manifest and data set are analyzed for compliance with system, user, and/or other requirements and, if compliant, added to the system for collaborative use as further described herein. The manifest and other metadata associated with the ingested data set may be stored within system 1000 for use in managing further access.

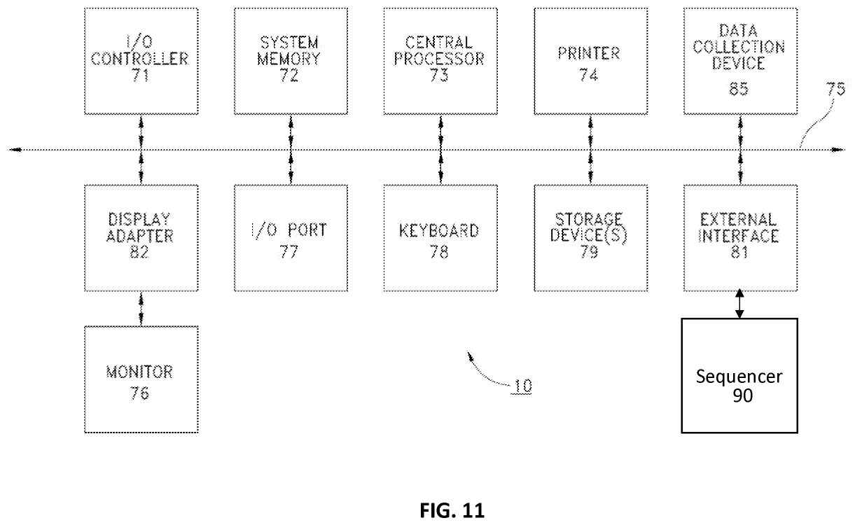

FIG. 11 illustrates an example computer system that may be utilized to implement techniques disclosed herein. Any of the computer systems mentioned herein, such as for hosting the systems and implementing the processes described for managing and storing data, may utilize any suitable number of subsystems. Examples of such subsystems are shown in FIG. 11 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones, telecommunication devices or other mobile devices. In some embodiments, a cloud infrastructure (e.g., Amazon Web Services), a graphical processing unit (GPU), etc., can be used to implement the disclosed techniques.

The subsystems shown in FIG. 11 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner.

The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A sequencer 90 (e.g., a nanopore sequencer) is connected through external interface 81 for providing sequencing data to a data collection device 85 and/or storage devices 79.

A computer system can include a plurality of the same components or subsystems, e.g., connected together by external interface 81 or by an internal interface. In some embodiments, computer systems, subsystems, or apparatuses can communicate over a network. In such instances, one computer can be considered a client and another computer a server, where each can be part of the same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Machine learning models utilized herein may include one or more of a Naïve Bayes (NB) model, a logistic regression (LR) model, a random forest (RF) model, a support vector machine (SVM) model, an artificial neural network model, a multilayer perceptron (MLP) model, a convolutional neural network (CNN), a Large Language model (LLM), and/or other machine learning or deep learning models, etc. The machine learning models can be updated/trained using a supervised learning technique, an unsupervised learning technique, etc.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

One or more aspects or features of the subject matter described herein can be realized in digital electronic circuitry, integrated circuitry, specially designed application-specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system for collaborative data analysis, comprising:
a project management engine configured to:
generate a plurality of project environments for analyzing a plurality of biomedical data sets of interest,
designate a set of authorized users with access to a given project environment of the plurality of project environments, and
provide access, in each of the plurality of project environments, to at least one data analysis engine for analyzing at least one of the plurality of biomedical data sets;
a data ingestion engine configured to:
receive a data manifest associated with at least one of the plurality of biomedical data sets of interest, the data manifest comprising information associated with the at least one biomedical data set of interest,
receive the at least one biomedical data set of interest,
ingest the at least one biomedical data set of interest based on the associated data manifest, and
store the ingested data of the data set of interest in a data storage system for access within at least one of the plurality of project environments; and
a user interface accessible by any of the authorized users, the user interface providing access to at least one of the project environments in order to cause analysis to be performed on at least one of the plurality of data sets of interest.

2. The system of claim 1, wherein ingesting the at least one biomedical data set of interest comprises validating that metadata associated with the data set of interest corresponds to metadata identified in the data manifest.

3. The system of claim 2, wherein, upon validating the metadata associated with the data set of interest corresponds to metadata identified in the data manifest, the ingestion engine is configured to enrich the at least one biomedical data set of interest by assigning a unique identifier to the biomedical data set of interest, the unique identifier being usable to track the use of the at least one biomedical data set of interest within the project management engine.

4. The system of claim 1, wherein:
the at least one data analysis engine is configured to perform analysis on at least one of the plurality of biomedical data sets, generate output indicative of the analysis, and store an updated data set based on the generated output; and
the at least one data ingestion engine is configured to ingest the updated data set and store the ingested updated data set in the data storage system for access within at least one of the plurality of project environments.

5. The system of claim 4, wherein each of the plurality of project environments comprises a controlled sub-environment comprising high performance computing system and an uncontrolled sub-environment comprising data analytics computing system.

6. The system of claim 1, wherein each of the plurality of biomedical data sets is assigned to at least one data schema based on the data manifest, and wherein the system further comprises a data management module configured to manage the data schema existing in the system.

7. The system of claim 6, wherein the data management module is configured to create additional data schema, modify existing data schema, and track modifications of existing data schema as different versions of the schema.

8. A system for collaborative and secure biomedical data processing, comprising:
a secure database system configured for cataloging and controlling secure access to sets of biomedical data;
one or more processors programmed and configured to:
receive a biomedical data set and a request to ingest the biomedical data set for storing and cataloging in the secure database system;
receive a data manifest associated with the biomedical data set, the manifest comprising a type of biomedical data of the biomedical data set and biological attributes pertaining to one or more subjects of the biomedical data set;
determine that the biomedical data set is validated by comparing properties of the biomedical data set and data manifest with one or more validation requirements for ingesting a biomedical data set having properties identified in the received manifest;
in response to validating the received biomedical data set, cataloging and securing the biomedical data set and associated manifest in the secure biomedical database system;
receive a request to generate a project environment of at least one project type of a plurality of project types for the received biomedical data set;
in response to receiving the request, generating an instance of a secure system project environment, the project environment providing particular types of data access and control to the biomedical data set catalogued in the secure database system based on the at least one project type and based on authenticated credentials of one or more system users requesting access;
receive a request to access the catalogued biomedical data set from a system user, the request associated with the instance of the generated project environment;
in response to the request for access and based on a validation of credentials of the system user, creating an instance of a secure data pipeline through which the requesting system user is granted types of data access and control to the catalogued and secure biomedical data set.

9. The system of claim 8 wherein the granting of particular types of data access and control to the biomedical data set is based on privacy or security rules applicable to a geographic region within which the data set is stored or from where the data set is accessed.

10. The system of claim 8 wherein the at least one project type comprises a data analytics research project type and wherein the particular types of data access and control applied to the data set comprises limiting access to anonymized or pseudo-anonymized versions of the data set and to the creation of and/or viewing of data analysis or models.

11. The system of claim 8 wherein the at least one project type comprises a software development project type and wherein the particular types of data access and control applied to the data set comprises permitting a creation of and/or editing of a software product code integrating use of the data set.

12. The system of claim 11 wherein the development of software product code comprises a development and/or training of a machine learning model.

13. The system of claim 8, wherein each of the plurality of project environments comprises a designated data analysis engine and dedicated storage for storing copies of the ingested data.

14. The system of claim 8, further comprising a data catalog listing ingested data sets of interest and information associated with the ingested data sets of interest.

15. A method for collaborative and secure biomedical data processing, the method comprising:
receiving a biomedical data set and a request to ingest the biomedical data set for storing and cataloging in a secure database system;
receiving a data manifest associated with the biomedical data set, the manifest comprising a type of biomedical data of the biomedical data set and biological attributes pertaining to one or more subjects of the biomedical data set;
determining that the biomedical data set is validated by comparing properties of the biomedical data set and data manifest with one or more validation requirements for ingesting a biomedical data set having properties identified in the received manifest;
in response to validating the received biomedical data set, cataloging and securing the biomedical data set and associated manifest in the secure biomedical database system;
receiving a request to generate a project environment of at least one project type of a plurality of project types for the received biomedical data set;
in response to receiving the request, generating an instance of a secure system project environment, the project environment granting particular types of data access and control to the biomedical data set catalogued in the secure database system based on the at least one project type and based on authenticated credentials of one or more system users requesting access;
receiving a request to access the catalogued biomedical data set from a system user, the request associated with the instance of the generated project environment;
in response to the request for access and based on a validation of credentials of the system user, creating an instance of a secure data pipeline through which the requesting system user is granted types of data access and control to the catalogued and secure biomedical data set.

16. The method of claim 15 wherein granting particular types of data access and control to the biomedical data set is based on privacy or security rules applicable to a geographic region within which the data set is stored or from where the data set is accessed.

17. The method of claim 15 wherein the at least one project type comprises a data analytics research project type and wherein the particular types of data access and control applied to the data set comprises limiting access to anonymized or pseudo-anonymized versions of the data set and to the creation of and/or viewing of data analysis or models.

18. The method of claim 15 wherein the at least one project type comprises a software development project type and wherein the particular types of data access and control applied to the data set comprises the creation of and/or editing of a development of software product code integrating use of the data set.

19. The method of claim 18 wherein the development of software product code comprises a development and/or training of a machine learning model.
